(19) United States
(12) Patent Application Publication
(10) Pub. No.: US 2023/0321032 A1
(43) Pub. Date: Oct. 12, 2023

(54) FUNCTIONALIZED MESOPOROUS SILICA NANOPARTICLES FOR TREATMENT OF PERIODONTAL DISEASE

(71) Applicant: University of Kentucky Research Foundation, Lexington, KY (US)

(72) Inventors: Stephen E. Rankin, Lexington, KY (US); Barbara L. Knutson, Lexington, KY (US); Octavio A. Gonzalez Duque, Lexington, KY (US); Luciana M. Shaddox, Lexington, KY (US); John M. Littleton, Lexington, KY (US)

(21) Appl. No.: 18/296,616

(22) Filed: Apr. 6, 2023

Related U.S. Application Data
(60) Provisional application No. 63/328,086, filed on Apr. 6, 2022.

(51) Int. Cl. A61K 31/352; A61K 9/14; A61K 47/69; A61K 47/52; A61K 47/54; A61P 1/02

(52) U.S. Cl. CPC A61K 31/352; A61K 9/143; A61K 47/6929; A61K 47/52; A61K 47/54; A61P 1/02

(57) ABSTRACT

The present disclosure concerns mesoporous silica nanoparticles (MSNP) functionalized to cross in and out of cells and loaded with one or more therapeutics to provide a vehicle that can effectively provide localized treatment to both intracellular and extracellular space. The MSNPs provide a vehicle for adjunct treatment of periodontitis, being able to provide treatment against the microbes and to the tissue.

### CROSS-REFERENCE TO RELATED APPLICATIONS

This application claims priority to U.S. Provisional Patent Application 63/328,086, filed Apr. 6, 2022, the content of which is hereby incorporated by reference in its entirety.

### BACKGROUND

Periodontal disease is a common condition, marked by the microbial infection in spaces between the gums and teeth or periodontitis. Current lines of therapy are largely limited to reliance on the physical removal of the amassed infecting microbe(s), such as with the removal of a plaque, tartar, or biofilm. In some cases, surgery may be needed to access the site of infection and remove the source microbe(s).Current methodologies can also use adjunctive therapies, such as a systemic antibiotic or local therapies. Locally administered therapies to the periodontal pocket are also available, including gel-based therapies, such as ATRIDOX™, or microparticles based therapies, such as ARESTIN™. These adjunct therapies however suffer from some significant shortcomings. Systemic therapies do not readily reach the extra-tissue space where the infection is occurring. Locally administered therapies do not provide any tissue penetration, yet periodontal-pathogens will invade the oral epithelium. The partial effectiveness of localized therapy thus requires repeated retreatment and re-application. Accordingly, there is a need for a better mode of treatment that will allow for the removal of the microbe(s) while also helping the tissue respond.

### SUMMARY

In aspects, the present disclosure concerns a mesoporous silica nanoparticle (MSNP) comprised of silicon dioxide nanoparticles of 100 nm to 900 nm in diameter with pores of 2 nm to 50 nm width dispersed throughout, wherein one or more molecules of silicon dioxide is functionalized with an appended functional group.

In aspects, the diameter is of 100 nm to 200 nm. In aspects, the pores are of 2 nm to 10 nm in width. In aspects, the functional group is selected from a transitional metal oxide, an alkyl group, an aryl group, a sulfhydryl group, a carboxylate group, a chloropropyl group, a primary amide group, a diamine group, a triamine group, and a phenylboronic acid group.

In some aspects, the functional group comprises titanium dioxide.

In some aspects, the functional group comprises a primary amide.

In aspects, the MSNP is loaded with a therapeutic agent. In aspects, the therapeutic is loaded at a concentration density of 0.1 μg/mm² to 10 μg/mm². In some aspects, the therapeutic comprises an antibiotic. In some aspects, the therapeutic agent comprises an antifungal. In some aspects, the therapeutic agent comprises an antioxidant. In some aspects, the antioxidant comprises quercetin.

In aspects, one or more molecules of silicon dioxide are functionalized with a second functional group.

In some aspects, the diameter is 170 nm. In some aspects, the pore width is of 2.8 nm.

In aspects, the present disclosure concerns a method for treating periodontitis in a subject comprising administering a solution comprising the MSNP as disclosed herein suspended therein to a periodontal pocket in the subject. In aspects, the solution is applied to the periodontal pocket via a syringe.

In aspects, the present disclosure concerns a mesoporous silica nanoparticle (MSNP) of silicon dioxide nanoparticles of 170 nm in diameter with pores of 2.8 nm in width dispersed throughout, wherein one or more molecules of silicon dioxide is functionalized with an appended primary amide group. In some aspects, the MSNP further includes a titania functional group.

### BRIEF DESCRIPTION OF THE DRAWINGS

FIG. 1 shows an overview of a MSNPs filling a periodontal pocket. The inset shows a close-up microscopic image of an exemplary MSNP.

FIG. 2 shows a cell viability graph for treatment with MSNPs, MSNPs loaded with quercetin, and an ethanol control.

FIG. 3 shows a schematic of functionalizing the MSNPs with a fluorescent dye, RITC.

FIG. 4 shows cells four hours after treatment with ethanol, MSNPs or MSNPs functionalized with quercetin.

FIG. 5 shows a time-course comparison of 4, 8, 16, and 24 for treatment with MSNPs and MSNPs functionalized with quercetin.

FIG. 6 shows IL-8 measured levels following ethanol, quercetin or MSNPs functionalized with the shown amount of quercetin.

### DESCRIPTION

The present disclosure concerns functionalized mesoporous nanoparticles (MSNPs) for a localized treatment of the gingivae of a subject. The MSNPs of the present disclosure can penetrate both into a periodontal pocket, as well as into cells and tissue. The MSNPs of the present disclosure are readily dispersed within a solution, such as an aqueous solution. As such, a subject can apply the MSNPs such as through a rinse or the end of a syringe. The size of the MSNPs allows for their access to a periodontal pocket or a space between a tooth and the gingivae. That the MSNPs of the present disclosure can access the space and will be taken up by the surrounding tissue provides a first-line therapeutic approach to treating periodontitis.

In some aspects, the MSNPs are of an inert material. In some aspects, the MSNPs are of a silica base material or silicon dioxide (SiO₂). In aspects, the MSNPs are a silica nanoparticle (NP) with a cross-sectional distance or diameter of less than 1 micron, such as about 10, 50, 100, 150, 200, 250, 300, 350, 400, 500, 600, 700, 800, and 900 nm, or combinations thereof. In some aspects, the silica MSNPs are less than 500 nm in diameter. In some aspects, the MSNPs are a silica NP of about 100 to 200 nm in diameter, including 110, 120, 130, 140, 150, 160, 170, 180, and 190 nm. As set forth in the working examples, the NPs may be of about 170 nm in diameter.

In aspects, the silica MSNPs are treated to have pores on the surface and/or throughout the body of the NPs. In aspects, the silica NPs are mesoporous, or contain pores of 2 to 50 nm in diameter, including about 5, 10, 15, 20, 25, 30, 35, 40, and 45 nm. In some aspects, the pores are of less than 5 nm in diameter, of less than 4 nm in diameter, or of less than 3 nm in diameter, including about 2.1, 2.2, 2.3, 2.4, 2.5, 2.6, 2.7, 2.8, and 2.9 nm in diameter. As set forth in the working examples, the pores may be of about 2.8 nm in diameter. The MSNPs therefore possess a surface area on the order of 500-1200 m²/g. In aspects, the present disclosure provides mesoporous NPs with pore diameters between about 2 and 50 nm that are of silicon dioxide (silica) nanoparticles less than 1 micron in diameter. As set forth herein, the pore size can assist to regulate the rate of release of a loaded therapeutic or other small molecule contained within the pores. It is thus as aspect of the present disclosure that the MSNPs deliver a loaded therapeutic or small molecule at a desired rate and/or over a desired period of time.

In aspects, the silica MSNPs are functionalized various organic and inorganic groups to impart specific properties of use for delivery of chemotherapeutic compounds for treatment of periodontal disease, and potentially other inflammatory conditions. The presence of the oxygen within the silica provide an intermediary —OH group that can readily be used to attach functional groups to the surface and pore interiors of the MSNPs.

In aspects, the MSNPs are functionalized to assist in cell/tissue penetration. For example, the presence of a positive charge from appending an amine from an appended primary amide to the surface of the MSNPs allows for the MSNPs to penetrate cells. Similarly, the surface presence of transition metal oxides such as titania (TiO₂) selectively binds to flavonoids. Functionalization may also assist in loading the MSNPs with small molecule therapeutics. To assist loading and retention of poorly water soluble compounds, hydrophobicity can be introduced using alkyl groups (including but not limited to methyl, propyl, octyl, decyl, dodecyl) or aryl groups (such as phenyl, tolyl, and 4-methoxyphenyl). Complementary functionality to drug targets may be introduced into pores via functional groups including sulfhydryl, carboxylate, chloropropyl, diamine, triamine, and phenylboronic acid.

In aspects, the MSNPs of the present disclosure are functionalized with two or more different functional groups. The surface functionality inside and outside of the pores can be independently controlled to make the particles multifunctional. As shown in the working examples, positive charge at the exterior surface generated by functionalization with amine groups makes the particles cell penetrating. Functionalization of the pores with titanium dioxide (titania) allows for chelation of inflammatory compounds such as flavonoids. As demonstrated in the working examples, the functionalization allows for the movement into and out of cells. Furthermore, depending on the functionalizing group, the MSNPs of the present disclosure can offer a delivery mechanism that provides benefit intracellularly and extracellularly, while itself possessing a low toxicity to the tissue. The ability access both spaces and deliver a payload therein addresses many of the short-comings of traditional treatment for periodontitis. The present disclosure provides a highly effective therapy the reduces the likelihood of more treatments and more invasive future treatments being needed. The MSNPs are further biocompatible and generally recognized as safe in oral delivery applications, while also being nontoxic if absorbed in the bloodstream.

The MSNPs can be further loaded with one or more materials as a vehicle to deliver a high payload thereof in situ. In some aspects, the controlled pore size and high surface area allow for loading with small molecule drugs, such as anti-microbial therapeutics. Antimicrobial and antibiotic compounds (metronidazole, amoxicillin, etc.) can be loaded into the pore space using solvent evaporation, or functionalization with additional groups as needed. Further routes to controlling release by modifying the pore openings with responsive or biodegradable functionality can be utilized to further increase the effectiveness of the MSNP therapeutic. In some aspects, the MSNPs are loaded with a concentration density of the concentration density of from 0.1 μg/mm² to 10 μg/mm², including about 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, and 9.5 μg/mm² and all ranges therein.

In aspects, the MSNPs of the present disclosure are loaded with one or more therapeutic agents. As described herein, the functionalization of the MSNPs allows to the MSNPs to cross in and out of cells. As such, when applied to an extracellular space, such as a periodontal pocket, the MSNPs can interact with the cells and the microbes in the space. As such, the MSNPs can provide a benefit to the underlying tissue, as well as deliver anti-microbial treatment, such as with an antibiotic or an anti-fungal. In aspects, the therapeutic agent may include an antibiotic, such as a penicillin, a glycopeptide, a sulfonamide, a fluoroquinolone, a macrolide, a tetracycline, a cephalosporin, or a combination thereof. Examples include phenoxymethylpenicillin, penicillin, dicloxacillin, amoxicillin, ampicillin, nafcillin, oxacillin, penicillin V, penicillin G, cephalosporin, cefaclor, cefazolin, cefadroxil, cephalexin, cefoxime, cefixime, cefoxitin, ceftriaxone, tetracycline, doxycycline, minocycline, sarecycline, erythromycin, clarithromycin, azithromycin, fidaxomicin, roxithromycin, ciprofloxacin, ofloxacin, levofloxacin, moxifloxacin, sulfamethoxazole, sulfasalazine, sulfacetamide, sulfadiazine, vancomycin, dalbavancin, oritavancin, telavancin, or combinations thereof. In aspects, the therapeutic may include an anti-fungal such as metronidazole, nystatin, ketoconazole, fluconazole, miconazole, econazole, bifonazole, butoconazole, fenticonazole, isoconazole, clotrimazole, and amphotericin.

In aspects, the functionalized MSNPs may be loaded with an anti-oxidant agents. Examples of such include quercetin, retinol, L-ascorbate (Vitamin C), Vitamin E, rosemary, polyphenols, sage, glutathione, resveratrol, ethoxyquingallic acid, caffeic acid, p-coumeric acid, p-hydroxy benzoic acid, ferulic acid, chlorogenic acid, daidzin, glycitin, genistin, daidzein, genistein, flavonoids, isoflavonoids, neoflavonoids, tertbutylhydroquinone, lipoic acid, melatonin, tocopherols, tocotrienols, thiols, β-carotene, retinoic acid, and catechin.

As identified herein, the pore size may regulate the rater of release and/or the residency time of the therapeutic within the MSNP. As such, the loading density may be elevated in order to provide a longer course of therapeutic release. For example, narrowing the pore width and increasing the concentration density may allow for an increase in dose released and dose time course.

The present disclosure also includes methods of administering the MSNPs as described herein, methods of treating periodontitis by administering the MSNPs as described herein, and methods of preparing the MSNPs as described herein. In aspects, the MSNPs are prepared by functionalizing the silica through the SiOH intermediary and attaching the functional group through the oxygen. For example, as set forth in the examples, the MSNPs can be functionalized with TiO₂ and/or NH₂. The MSNPs can be loaded with one or more therapeutics by dissolving in a solution and forcing evaporation once filled in the pores of the MSNPs.

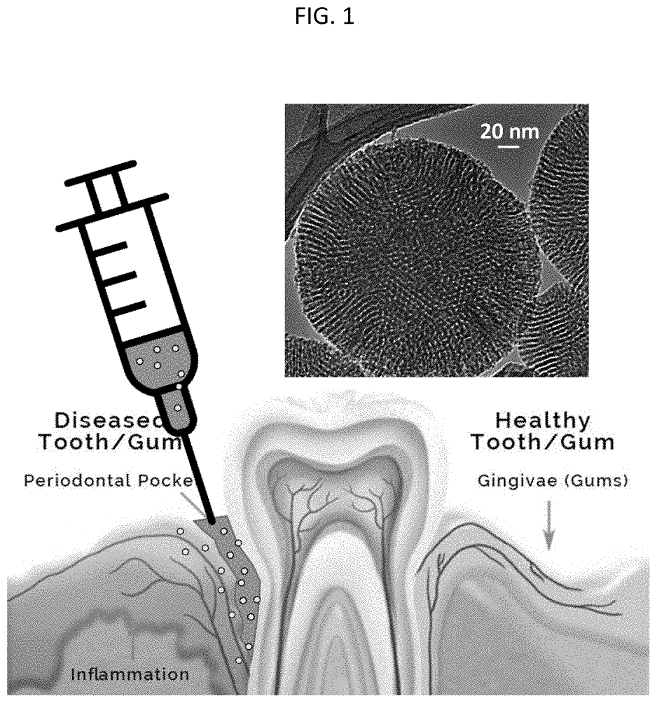

In aspects, the methods of the present disclosure include administering the MSNPs to an oral cavity. In some aspects, the MSNPs can be suspended within a solution and administered as an oral rinse. In other aspects, the suspended-solution can be forced through the barrel of a syringe and out of a needle operably attached thereto to site-specifically deliver the MSNPs, such as into a periodontal pocket (see FIG. 1). The MSNPs can be administered alone as an adjuvant property or as part of a further procedure, such as scraping a plaque or biofilm from a tooth surface and/or periodontal pocket. The MSNPs can be easily delivered as an adjuvant therapy for targeted delivery of antimicrobial compounds directly to epithelial cells in gum tissue affected by periodontitis. The versatility of the MSNPs also allows for simultaneous delivery of naturally occurring anti-inflammatory compounds (such as anti-oxidants) to reduce damage in infected tissue and promote healing.

### EXAMPLES

MSNPs of ˜170 nm in diameter with ˜2.8 nm pores were functionalized with 425 mg/g of TiO₂ or 0.0012 mmol/m² NH₂ and then assessed for effects on cell viability, cellular uptake, and inflammatory response. The test loaded agent was quercetin.

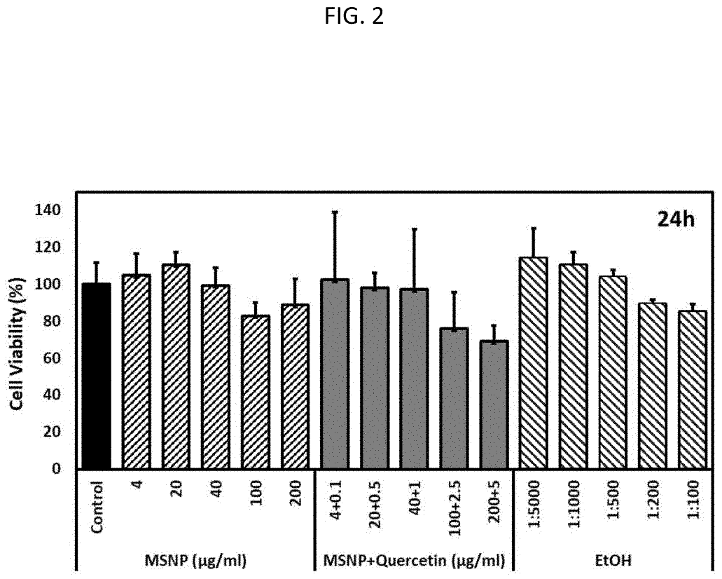

FIG. 2 sets forth the cytotoxicity studies with MSNPs alone, quercetin loaded MSNPs, and ethanol as a vehicle administered to human oral epithelial cells (OKF6) in vitro. After 24 hrs of exposure, cell viability was assessed by WST-1 cell assay.

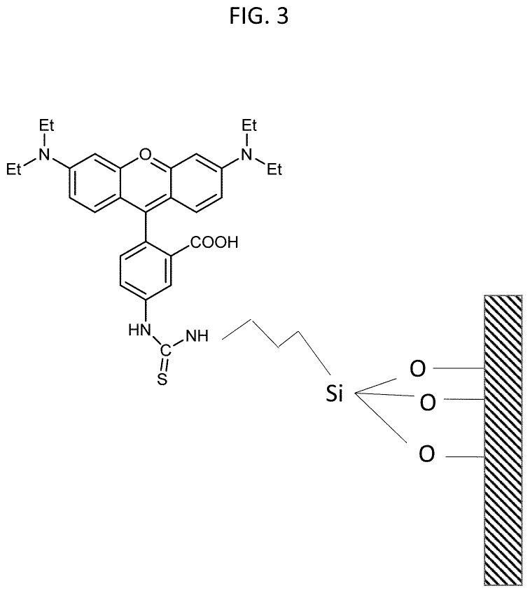

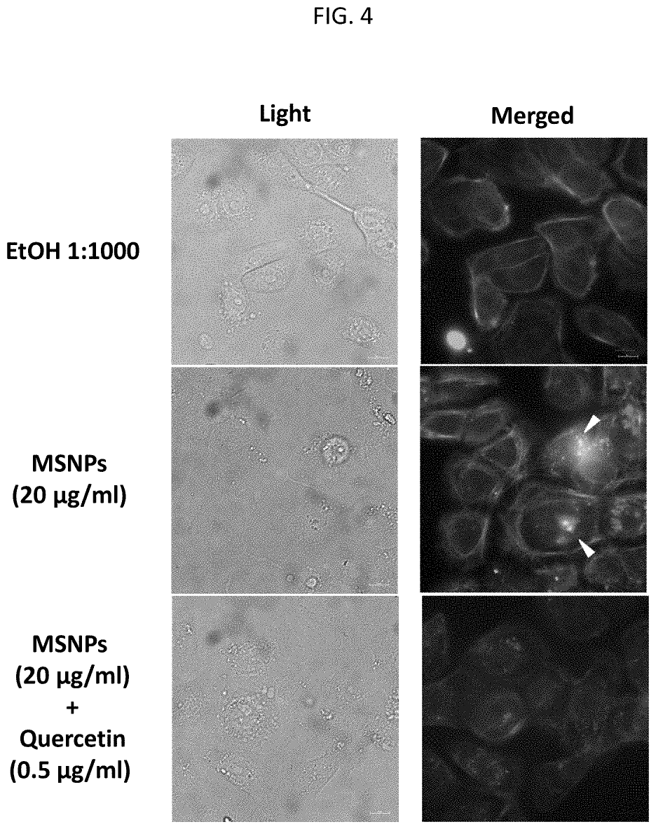

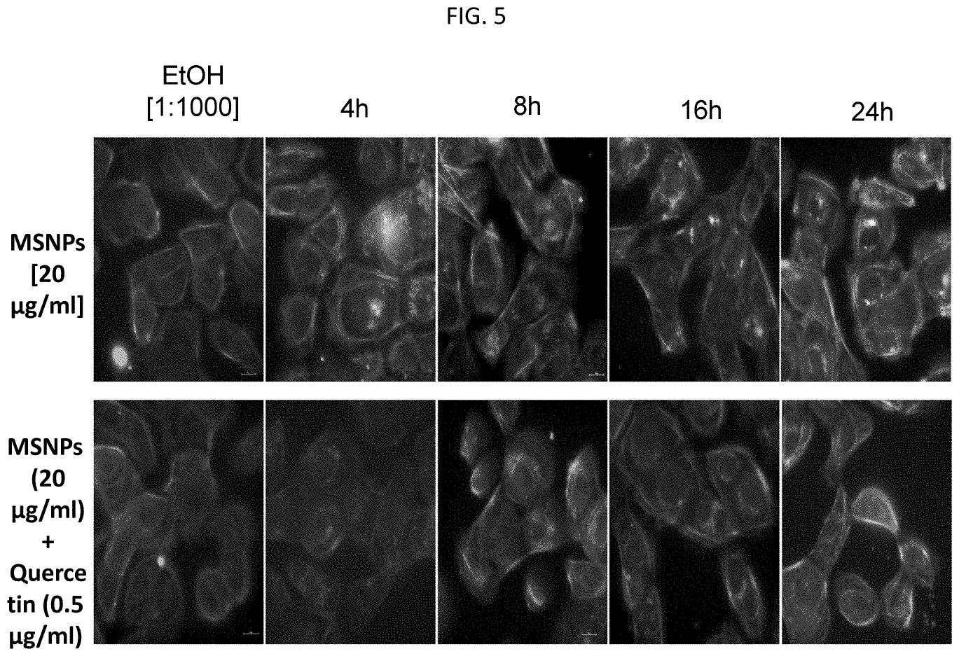

To assess for cell uptake, amine functionalized MSNPs were loaded with the fluorescent dye RITC (fluorescent rhodamine B isothiocyanate) as depicted in FIG. 3. OKF6 cells were assessed at 4, 8, 16, and 24 hours by F-actin staining, Hoechst staining and for the presence of the RITC by fluorescence microscopy. FIG. 4 shows the merged stainings of the F-actin, nucleus and RITC, identifying that the cells did uptake the MSNPs at 4 hours. FIG. 5 shows the merged images at all time points.

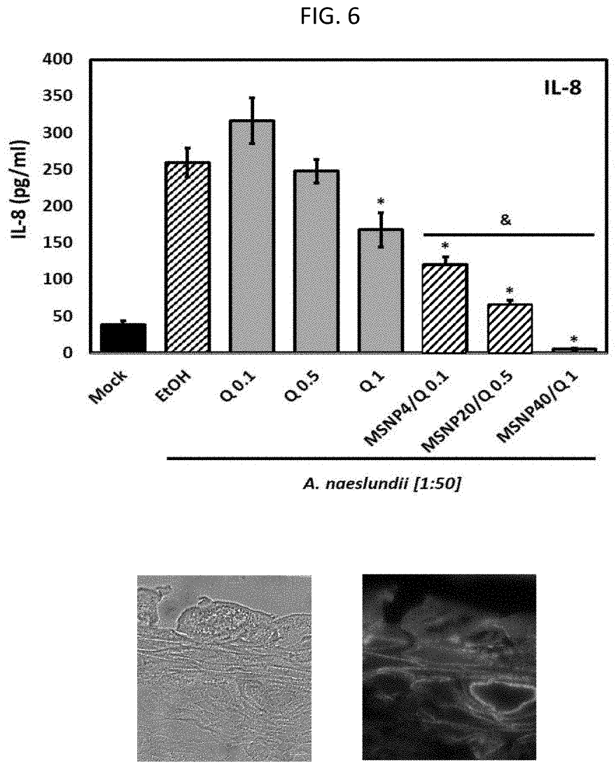

To assess for the effect of the MSNPs on inflammation response, OKF6 cells were challenged for 24 hours in the presence of Actinomyces naeslundii, a gram positive rod-shaped bacterium often found in the mouth of humans. FIG. 6 (upper graph) shows that the quercetin loaded MSNPs had a good effect on limiting the presence of IL-8, particularly in comparison to quercetin alone. The lower images of FIG. 6 show light and merged fluorescence, confirming the MSNPs were taken up.

Collectively, these data demonstrate that TiO₂ and NH₂ functionalized MSNPs can efficiently and rapidly be internalized by oral epithelial cells. Further, cell viability is not compromised and the attenuation of bacteria-induced pro-inflammatory responses is improved.

While particular aspects have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

We claim:

1. A mesoporous silica nanoparticle (MSNP) comprised of silicon dioxide nanoparticles of 100 nm to 900 nm in diameter with pores of 2 nm to 50 nm width dispersed throughout, wherein one or more molecules of silicon dioxide is functionalized with an appended functional group.

2. The MSNP of claim 1, wherein the diameter is of 100 nm to 200 nm.

3. The MSNP of claim 1, wherein the pores are of 2 nm to 10 nm in width.

4. The MSNP of claim 1, wherein the functional group is selected from a transitional metal oxide, an alkyl group, an aryl group, a sulfhydryl group, a carboxylate group, a chloropropyl group, a primary amide group, a diamine group, a triamine group, and a phenylboronic acid group.

5. The MSNP of claim 1, wherein the functional group comprises titanium dioxide.

6. The MSNP of claim 1, wherein the functional group comprises a primary amide.

7. The MSNP of claim 1, wherein the MSNP is loaded with a therapeutic agent.

8. The MSNP of claim 1, wherein the therapeutic is loaded at a concentration density of 0.1 μg/mm² to 10 μg/mm²,

9. The MSNP of claim 7, wherein the therapeutic comprises an antibiotic.

10. The MSNP of claim 7, wherein the therapeutic agent comprises an antifungal.

11. The MSNP of claim 7, wherein the therapeutic agent comprises an antioxidant.

12. The MSNP of claim 11, wherein the antioxidant comprises quercetin.

13. The MSNP of claim 1, wherein one or more molecules of silicon dioxide are functionalized with a second functional group.

14. The MSNP of claim 1, wherein the diameter is 170 nm.

15. The MSNP of claim 1, wherein the pore width is of 2.8 nm.

16. A method for treating periodontitis in a subject comprising administering a solution comprising the MSNP of claim 1 suspended therein to a periodontal pocket in the subject.

17. The method of claim 16, wherein the solution is applied to the periodontal pocket via a syringe.

18. A mesoporous silica nanoparticle (MSNP) comprised of silicon dioxide nanoparticles of 170 nm in diameter with pores of 2.8 nm in width dispersed throughout, wherein one or more molecules of silicon dioxide is functionalized with an appended primary amide group.

19. The MSNP of claim 18, further comprising a titania functional group.
